(19) United States
(12) Patent Application Publication
(10) Pub. No.: US 2005/0208084 A1
(43) Pub. Date: Sep. 22, 2005

(54) Use of alkylamidomandelates as flavourings

(76) Inventors: Jakob P. Ley, Holzminden (DE); Gerhard Krammer, Holzminden (DE); Arnold Machinek, Holzminden (DE)

Correspondence Address: ROYLANCE, ABRAMS, BERDO & GOODMAN, L.L.P., 1300 19TH STREET, N.W., SUITE 600, WASHINGTON,, DC, 20036, US

(21) Appl. No.: 10/517,197

(22) PCT Filed: Jun. 14, 2003
(86) PCT No.: PCT/EP03/06292

§ 371 Date: Dec. 8, 2004

(30) Foreign Application Priority Data
Jun. 17, 2002 (DE) 102 26 942.4

(51) Int. Cl. A61K 31/165; A61K 7/00

(52) U.S. Cl. 424/401; 514/621; 564/167

(57) ABSTRACT

The present invention describes various mandelic acid alkylamides and their use as pungent compounds and flavour compounds with a heat-generating effect, preferably in preparations for use in nutrition, oral hygiene or consumed for pleasure.

The invention relates to the use of mandelic acid alkylamides as flavour compounds, particularly as pungent compounds and flavour compounds with a heat-generating effect, in preparations for use in nutrition, oral hygiene or consumed for pleasure. The invention also relates to preparations for use in nutrition, oral hygiene or consumed for pleasure containing the mandelic acid alkylamides according to the invention, and to processes for the production of the preparations according to the invention.

Capsaicin [N-(4-hydroxy-3-methoxybenzyl)-8-methyl-(6E)-nonenoamide, cf. structure 1, FIG. 1] and other capsaicinoids have been known since 1871 as pungent-tasting and heat-generating flavour compounds from various varieties of capsicum, especially chilli. At an appropriately low dose of the capsaicinoids (the threshold value lies at a dilution of approx. 1:10⁵), only a pleasant, neutral pungency and a sensation of heat in the mouth are perceived. A problem with capsaicin lies in its high acute toxicity (LD₅₀ (mouse oral) 47 mg), which creates difficulties for its use in formulation, together with the chronic gastritis, kidney and liver damage (Römpp Lexikon Naturstoffchemie, Thieme 1997, p. 109) occurring in the case of frequent use and overdose. Thus, despite the good sensory properties, there is a need for less problematic pungent compounds. While it is true that piperine (1-piperoylpiperidine, cf. structure 2, FIG. 1), which occurs in white pepper, also produces a strong impression of pungency (Römpp Lexikon Naturstoffchemie, Thieme 1997, p. 500), it displays a relative pungency of only approx. 1% compared with capsaicin. Moreover, piperine possesses an intense inherent flavour reminiscent of pepper, and so it can be used only to a limited extent in many preparations.

[image]

The object of the present invention was to find substances having a pungent and/or heat-generating effect and a neutral flavour profile and capable of being used as flavour compounds in preparations for use in nutrition, oral hygiene or consumed for pleasure.

The invention therefore relates to the use of mandelic acid alkylamides of the general formula (I) 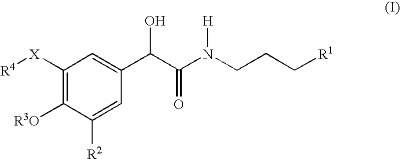
wherein
X represents a single bond or an oxygen atom and
R¹ represents a linear or branched alkyl residue with 1 to 20 carbon atoms or a linear or branched alkenyl residue with 2 to 20 carbon atoms and
R² represents a hydrogen atom, a hydroxy group or an O—R⁵ group and
R³, R⁴ and R⁵, independently of one another, represent hydrogen or a lower alkyl residue or a lower alkenyl residue or
R³ and R⁴ together represent a —CR⁶R⁷— group and
R⁶ and R⁷, independently of one another, represent hydrogen or lower alkyl residues or lower alkenyl residues,
   and the various stereoisomers or mixtures thereof as flavour compounds, preferably as pungent compounds or flavour compounds with a heat-generating effect, particularly preferably as pungent compounds or flavour compounds with a heat-generating effect in preparations for use in nutrition, oral hygiene or consumed for pleasure. Heat-generating substances or substances with a heat-generating effect are intended to mean those creating a sensory impression of heat.

A lower alkyl residue consists of 1 to 5 carbon atoms and can be, for example: methyl, ethyl, 1-propyl, 2-propyl, 1-butyl, 2-butyl, tert.-butyl, 2-methylprop-1-yl, 1-, 2- or 3-pentyl, 2-methylbut-1-yl, 2-methylbut-2-yl, 3-methylbut-1-yl or 3-methylbut-2-yl.

A lower alkenyl residue consists of 2 to 5 carbon atoms and can be, for example: ethenyl, prop-2-en-1-yl, prop-1-en-1-yl, prop-1-en-2-yl, 1- or 2-cyclopropenyl, but-1-en-1-yl, but-1-en-2-yl, but-1-en-3-yl, but-2-en-1-yl, but-3-en-1-yl, but-2-en-2-yl, 2-methylprop-1-en-1-yl, 2-methylprop-2-en-1-yl, 1,3-butadien-1-yl, 1,3-butadien-2-yl, pent-1-en-1-yl, pent-1-en-2-yl, pent-1-en-3-yl, pent-1-en-4-yl, pent-2-en-1-yl, pent-2-en-2-yl, pent-2-en-3-yl, pent-2-en-4-yl, pent-3-en-1-yl, pent-4-en-1-yl, 1,3-pentadien-1-yl, 1,3-pentadien-2-yl, 1,3-pentadien-3-yl, 2,4-pentadien-2-yl, 2,4-pentadien-1-yl, 1,4-pentadien-1-yl, 1,4-pentadien-2-yl, 1,4-pentadien-3-yl, 1,2- or 3-cyclopentenyl, 1-,2- or 3-cyclopentadienyl, 3-methylbut-1-en-1-yl, 3-methylbut-1-en-2-yl, 3-methylbut-1-en-3-yl, 3-methylbut-1-en-4-yl, 3-methylbut-2-en-1-yl, 3-methylbut-2-en-2-yl, 3-methylbut-2-en-4-yl, 2-methylbut-1-en-1-yl, 2-methylbut-1-en-3-yl, 2-methylbut-1-en-4-yl, 3-methylidenebut-1-yl, 2-methyl-1,3-butadien-1-yl, 2-methyl-1,3-butadien-3-yl, 2-methyl-1,3-butadien-4-yl, 2-methylidenebut-3-en-1-yl and the Z and E isomers of the above-mentioned residues that may be possible in each case.

A linear or branched alkyl residue consists of 1 to 20 carbon atoms and can be, for example: methyl, ethyl, 1-propyl, 2-propyl, 1-butyl, 2-butyl, tert.-butyl, 2-methylprop-1-yl, 1-, 2- or 3-pentyl, 2-methylbut-1-yl, 2-methylbut-2-yl, 3-methylbut-1-yl or 3-methylbut-2-yl, 1-, 2- or 3-hexyl, 1-, 2-, 3- or 4-heptyl, 1-, 2-, 3- or 4-octyl, 1-, 2-, 3- or 5-nonyl, or 1-, 2-, 3-, 4- or 5-decyl or the various isomers of undecyl, dodecyl, tridecyl, tetradecyl, pentadecyl, hexadecyl, heptadecyl, octadecyl, nonadecyl, eicosyl.

A linear or branched alkenyl residue consists of 2 to 20 carbon atoms and can be, for example: ethenyl, prop-2-en-1-yl, prop-1-en-1-yl, prop-1-en-2-yl, but-1-en-1-yl, but-1-en-2-yl, but-1-en-3-yl, but-2-en-1-yl, but-3-en-1-yl, but-2-en-2-yl, 2-methylprop-1-en-1-yl, 2-methylprop-2-en-1-yl, 1,3-butadien-1-yl, 1,3-butadien-2-yl, pent-1-en-1-yl, pent-1-en-2-yl, pent-1-en-3-yl, pent-1-en-4-yl, pent-2-en-1-yl, pent-2-en-2-yl, pent-2-en-3-yl, pent-2-en-4-yl, pent-3-en-1-yl, pent-4-en-1-yl, 1,3-pentadien-1-yl, 1,3-pentadien-2-yl, 1,3-pentadien-3-yl, 2,4-pentadien-2-yl, 2,4-pentadien-1-yl, 1,4-pentadien-1-yl, 1,4-pentadien-2-yl, 1,4-pentadien-3-yl, 3-methylbut-1-en-1-yl, 3-methylbut-1-en-2-yl, 3-methylbut-1-en-3-yl, 3-methylbut-1-en-4-yl, 3-methylbut-2-en-1-yl, 3-methylbut-2-en-2-yl, 3-methylbut-2-en-4-yl, 2-methylbut-1-en-1-yl, 2-methylbut-1-en-3-yl, 2-methylbut-1-en-4-yl, 2-methylidenbut-1-yl, 2-methyl-1,3-butadien-1-yl, 2-methyl-1,3-butadien-3-yl, 2-methyl-1,3-butadien-4-yl, 2-methylidenebut-3-en-1-yl as well as all possible alkenes, alkadienes, alkatrienes, alkatetraenes and alkapentaenes of C₆ to C₂₀ and the Z and E isomers of the above-mentioned residues that may be possible in each case.

Surprisingly, upon sensory investigation the mandelic acid alkylamides according to the invention display a pleasant, strongly pungent and hot taste impression, which is neutral and relatively long-lasting.

The present invention also provides preparations, semi-finished products and odour, flavour and taste compositions containing these compounds.

The mandelic acid alkylamides according to the invention can naturally also be used in cosmetic or dermatological preparations for the production of heat on the skin.

Preferred is the use of mandelic acid alkylamides of general formula (I) 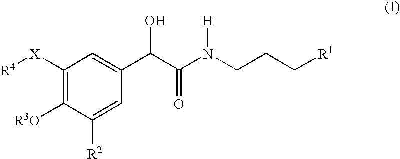
wherein
X represents a single bond or an oxygen atom and
R¹ represents a linear or branched alkyl residue with 1 to 10 carbon atoms or a linear or branched alkenyl residue with 2 to 10 carbon atoms and
R² represents a hydrogen atom, and
R³ and R⁴, independently of one another, represent hydrogen or a lower alkyl residue or a lower alkenyl residue,
   and the various isomers or mixtures thereof as flavour compounds, preferably as pungent compounds and flavour compounds with a heat-generating effect, particularly preferably as pungent compounds and flavour compounds with a heat-generating effect in preparations for use in nutrition, oral hygiene or consumed for pleasure, is preferred.

Particularly preferred is the use of mandelic acid alkylamides of general formula (I) 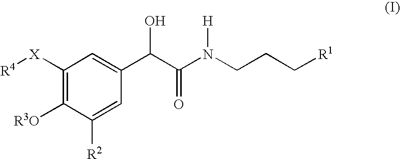
wherein
R¹ represents a linear or branched alkyl residue with 1 to 10 carbon atoms or a linear or branched alkenyl residue with 2 to 10 carbon atoms and
R² represents a hydrogen atom, and either
X represents a single bond and R³ and R⁴ both represent hydrogen (4-hydroxymandelic acid alkylamides) or
X represents a single bond, R³ a methyl group and R⁴ hydrogen (4-methoxymandelic acid alkylamides) or
X represents an oxygen atom and R³ and R⁴ both represent hydrogen (3,4-dihydroxymandelic acid alkylamides) or
X represents an oxygen atom and R³ hydrogen and R⁴ methyl (vanillomandelic acid alkylamides) or
X represents an oxygen atom and R⁴ hydrogen and R³ methyl (isovanillo-mandelic acid alkylamides),
   and the various stereoisomers or mixtures thereof as flavour compounds, preferably as pungent compounds or flavour compounds with a heat-generating effect, particularly preferably as pungent compounds or flavour compounds with a heat-generating effect in preparations for use in nutrition, oral hygiene or consumed for pleasure.

Especially preferred is the use of the compounds
2-(4-hydroxyphenyl)-2-hydroxy-N-heptylacetamide,
2-(4-hydroxyphenyl)-2-hydroxy-N-octylacetamide,
2-(4-hydroxyphenyl)-2-hydroxy-N-nonylacetamide,
2-(4-methoxyphenyl)-2-hydroxy-N-heptylacetamide,
2-(4-methoxyphenyl)-2-hydroxy-N-octylacetamide,
2-(4-methoxyphenyl)-2-hydroxy-N-nonylacetamide,
2-(3,4-dihydroxyphenyl)-2-hydroxy-N-octylacetamide,
2-(3-hydroxy-4-methoxyphenyl)-2-hydroxy-N-heptylacetamide,
2-(3-hydroxy-4-methoxyphenyl)-2-hydroxy-N-octylacetamide,
2-(3-hydroxy-4-methoxyphenyl)-2-hydroxy-N-nonylacetamide,
2-(4-hydroxy-3-methoxyphenyl)-2-hydroxy-N-heptylacetamide,
2-(4-hydroxy-3-methoxyphenyl)-2-hydroxy-N-octylacetamide,
2-(4-hydroxy-3-methoxyphenyl)-2-hydroxy-N-nonylacetamide, or
2-(4-hydroxy-3-methoxyphenyl)-2-hydroxy-N-(7-methyl-1-octyl)acetamide
   as flavour compounds, preferably as pungent compounds and flavour compounds with a heat-generating effect, particularly preferably as pungent compounds and flavour compounds with a heat-generating effect in preparations for use in nutrition, oral hygiene or consumed for pleasure.

The various mandelic acid alkylamides according to the invention, the stereoisomers and salts thereof can naturally be used according to the invention each individually or as mixtures.

The salts of the mandelic acid alkylamides according to the invention can be present as monovalent or optionally multivalent phenolate salts with inorganic cations. The cations of lithium, sodium, potassium, the ammonium ion, the cations of magnesium, calcium and strontium or the cations of aluminium, zinc, copper, iron or manganese are preferred.

2-(4-Hydroxy-3-methoxyphenyl)-2-hydroxy-N-octylacetamide (vanillomandelic acid octylamide) was described as an analgesic in J. Med. Chem., vol. 36, 1993, pages 2373 ff. However, no sensory evaluation of the substance or description of a use has been described. No other long-chain 4-hydroxymandelic acid alkylamides, vanillomandelic acid alkylamides and isovanillic acid alkylamides within the meaning of the invention are known. 3,4-Dihydroxymandelic acid alkylamides were described in DE-A 100 30 880 as antioxidants.

In particular, there has been no description in the literature of the use of the compounds according to the invention as flavour compounds or their use as pungent-tasting flavour compounds or as substances with a heat-generating effect.

Mandelic acid alkylamides of general formula (I) 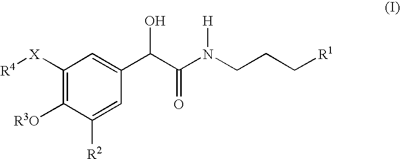
wherein
R¹ represents a linear or branched alkyl residue with 1 to 20 carbon atoms or a linear or branched alkenyl residue with 2 to 20 carbon atoms and
R² represents a hydrogen atom, and
   either
X represents a single bond,
R³ a lower alkyl residue or a lower alkenyl residue and
R⁴ hydrogen or
X represents an oxygen atom,
R³ hydrogen and
R⁴ a lower alkyl residue or a lower alkenyl residue or
X represents an oxygen atom,
R³ a lower alkyl residue or a lower alkenyl residue and
R⁴ hydrogen
   and the various stereoisomers or mixtures thereof, with the exception that X represents an oxygen atom, R¹ 1-pentyl, R² and R³ hydrogen and R⁴ methyl, are novel.

Especially preferred are mandelic acid alkylamides of general formula (I), wherein
R¹ represents a linear or branched alkyl residue with 1 to 10 carbon atoms or a linear or branched alkenyl residue with 2 to 10 carbon atoms, with the exception of R¹ representing 1-pentyl, and
R² represents a hydrogen atom, and
   either
X represents a single bond,
R³ a hydrogen atom or a methyl group and
R⁴ a hydrogen atom or
X represents an oxygen atom,
R³ hydrogen and
R⁴ a methyl residue or
X represents an oxygen atom,
R³ a methyl and
R⁴ hydrogen,
   and the various stereoisomers or mixtures thereof, with the exception that X represents an oxygen atom, R¹ 1-pentyl, R² and R³ hydrogen and R⁴ methyl.

Especially preferred are the compounds
2-(4-hydroxyphenyl)-2-hydroxy-N-heptylacetamide,
2-(4-hydroxyphenyl)-2-hydroxy-N-octylacetamide,
2-(4-hydroxyphenyl)-2-hydroxy-N-nonylacetamide,
2-(4-methoxyphenyl)-2-hydroxy-N-heptylacetamide,
2-(4-methoxyphenyl)-2-hydroxy-N-octylacetamide,
2-(4-methoxyphenyl)-2-hydroxy-N-nonylacetamide,
2-(3-hydroxy-4-methoxyphenyl)-2-hydroxy-N-heptylacetamide,
2-(3-hydroxy-4-methoxyphenyl)-2-hydroxy-N-nonylacetamide,
2-(4-hydroxy-3-methoxyphenyl)-2-hydroxy-N-heptylacetamide,
2-(4-hydroxy-3-methoxyphenyl)-2-hydroxy-N-nonylacetamide, and
2-(4-hydroxy-3-methoxyphenyl)-2-hydroxy-N-(7-methyl-1-octyl)acetamides.
   The mandelic acid alkylamides of general formula (I) 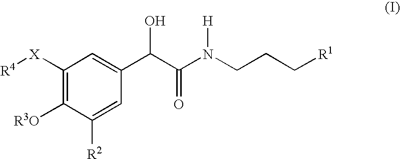
   wherein
R¹ represents a linear or branched alkyl residue with 1 to 20 carbon atoms or a linear or branched alkenyl residue with 2 to 20 carbon atoms and
R² represents a hydrogen atom, and
   either
X represents a single bond,
R³ and R⁴ represent hydrogen atoms or
X represents an oxygen atom,
R³ hydrogen and
R⁴ a lower alkyl residue or a lower alkenyl residue or
X represents an oxygen atom,
R³ a lower alkyl residue or a lower alkenyl residue and
R⁴ hydrogen
   and the various stereoisomers or mixtures thereof, with the exception that R¹ represents 1-pentyl, R² and R³ hydrogen and R⁴ methyl,
can be produced in that a mandelic acid of general formula II 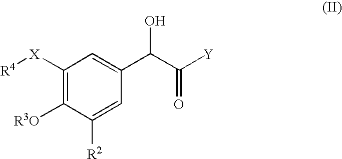
   wherein
X, R², R³ and R⁴ have the above meaning, and
Y represents an activated nucleofuge,
   or derivatives, the OH groups of which are protected with protective groups, are reacted with an alkylamine of general formula (IIIa) 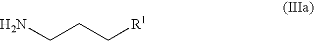
   or an alkylammonium salt of general formula (IIIb) 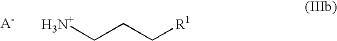
   wherein
R¹ has the meaning given above and
A⁻ denotes an inorganic or organic anion, e.g. halide, sulfate, hydrogen sulfate or acetate,
   optionally in the presence of solvents and auxiliary bases, and the protective groups of the OH groups are optionally split off.

Activated nucleofuges Y are e.g. the halides, preferably chloride, O-acyl residues, preferably O-acetyl, O-oxalyl or a residue of general formula II with Y as oxygen, in which case symmetric or mixed acid anhydrides are obtained, or O—R⁶ residues, wherein R⁶ represents lower alkyl residues, optionally substituted phenols, preferably mono-, di- or trinitrosubstituted phenols, nitrogen-containing N-hydroxyheterocycles, preferably N-hydroxysuccinimide, N-hydroxyphthalimide or N-hydroxybenzotriazole.

Acyl, carbamate or ether groups, e.g. acetyl, benzoyl, methoxycarbonyl, allyloxycarbonyl, methoxymethyl, methoxyethoxymethyl, tert.-butoxycarbonyl, allyl or benzyl groups, are preferably used as protective groups.

Protic or non-protic, polar and non-polar solvents, preferably water, ketones, alcohols, alkyl esters of aliphatic acids, chlorinated hydrocarbons, ethers or N-methylamides, can be used as the solvents. Water, ethanol, methanol, acetone, 1,4-dioxane, N-methylpyrrolidone, N,N-dimethylformamide, tetrahydrofuran, ethyl acetate, chloroform or mixtures of the last-mentioned solvents are particularly preferred.

As auxiliary bases, ammonium, alkali metal or alkaline earth metal carbonates, hydrogen carbonates or hydroxides, tertiary aliphatic amines, e.g. triethylamine, and inorganic or organic basic ion exchangers can, for example, be used.

The mandelic acid alkylamides according to the invention are particularly preferably produced from mandelic acid-N-hydroxysuccinimidyl esters, optionally blocked on the hydroxy groups with acetyl or methoxycarbonyl groups, with alkylamines of formula IIIa or the alkylammonium salts of formula IIIb in an aqueous solvent mixture, preferably a water/1,4-dioxane or water/acetone mixture, with one of the above-mentioned auxiliary bases at 5 to 100° C. The mandelic acid-N-hydroxysuccinimidyl esters are advantageously prepared from the corresponding free acid and N-hydroxysuccinimide (NHOSu) by means of a carbodiimide, preferably N,N′-dicyclohexylcarbodiimide (DCC) or diisopropylcarbodiimide (DIIC), in an aprotic solvent, preferably 1,4-dioxane, diethyl ether, tert.-butyl methyl ether, ethyl acetate or tetrahydrofuran, at 0 to 50° C., preferably at 5 to 30° C., the dissolved crude product separated from the residue by filtration and the filtrate directly reacted within the meaning of the invention with the alkylamines of formula IIIa or the alkylammonium salts of formula IIIb present in water or a water/1,4-dioxane or water/acetone mixture and one of the above-mentioned auxiliary bases. The process is illustrated by the following diagram, using 2-(4-hydroxy-3-methoxyphenyl)-N-nonyl-2-hydroxyacetamide as an example:

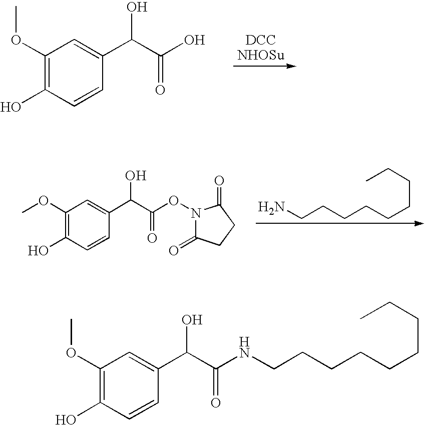

In particular, n-heptylamine, n-octylamine, n-nonylamine, 7-methyloctylamine or their respective ammonium salts are used as the alkylamines.

4-Hydroxymandelic acid, 4-methoxymandelic acid, 4-hydroxy-3-methoxymandelic acid or 3-hydroxy-4-methoxymandelic acid, the stereoisomers and the mixtures thereof are preferably used as the mandelic acids.

The mandelic acid alkylamides according to the invention can, however, also be obtained by direct condensation of the free acids of general formula II,
wherein X, R², R³ and R⁴ have the meanings given above and Y represents hydroxy,
with an alkylamine of general formula IIIa, wherein the residue R¹ has the meaning given above,
with or without solvents.

The reaction is illustrated in the following diagram using 2-(3-hydroxy-4-methoxyphenyl)-N-heptyl-2-hydroxyacetamide as an example:

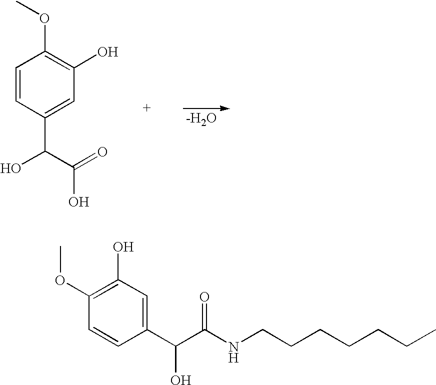

As the condensation agents, for example carbodiimides, preferably N,N′-dicyclohexylcarbodiimide, N,N′-diisopropylcarbodiimide or N,N′-carbonyl-diimidazole, and as the solvent, preferably aprotic solvents, e.g. 1,4-dioxane, diethyl ether, tert.-butyl methyl ether, ethyl acetate or tetrahydrofuran, can be used.

The mandelic acid alkylamides according to the invention are obtained from these reaction mixtures by purification steps that are known per se. It is advantageous to treat a solution of the mandelic acid alkylamides according to the invention in a water-immiscible solvent, e.g. ethyl acetate, chloroform, methylene chloride, aliphatic or aromatic hydrocarbons, diethyl ether or tert.-butyl methyl ether, with a mineral acid, e.g. dilute or concentrated hydrochloric acid, sulfuric acid, phosphoric acid, or an acidic, inorganic or organic ion exchanger to remove residues of the amine of general formula IIIa or the ammonium salt IIIb used.

Any protective groups still present must be removed using methods that are known per se.

In a particularly preferred embodiment of the invention, the mandelic acid alkylamides according to the invention are used in combination with other pungent-tasting and/or heat-generating substances or pungent-tasting plant extracts. In this way, a particularly rounded sensory profile can be achieved. In particular the combination of one or more of the mandelamides according to the invention with a pungent-tasting plant extract in a ratio of 0.01:1 to 100:1, preferably 0.1:1 to 10:1, produces a pleasant sensory profile.

Other pungent-tasting and/or heat-generating substances can be e.g. capsaicin, dihydrocapsaicin, gingerol, paradols, shogaols, piperine, carboxylic acid-N-vanillylamides, especially nonanoic acid-N-vanillylamide, 2-alkenoamides, especially 2-nonenoic acid-N-isobutylamide, cis- or trans-pellitorine or spilanthol, 2-nonenoic acid-N-4-hydroxy-3-methoxyphenylamide, alkyl ethers of 4-hydroxy-3-methoxybenzyl alcohol, especially 4-hydroxy-3-methoxybenzyl-n-butyl ether, alkyl ethers of 4-acyloxy-3-methoxybenzyl alcohol, especially 4-acetyloxy-3-methoxybenzyl-n-butyl ether and 4-acetyloxy-3-methoxybenzyl-n-hexyl ether, alkyl ethers of 3-hydroxy-4-methoxybenzyl alcohol, alkyl ethers of 3,4-dimethoxybenzyl alcohol, alkyl ethers of 3-ethoxy-4-hydroxybenzyl alcohol, alkyl ethers of 3,4-methylenedioxybenzyl alcohol, (4-hydroxy-3-methoxyphenyl)acetamides, especially (4-hydroxy-3-methoxyphenyl)acetic acid-N-n-octylamide, ferulic acid phenethyl-amides, nicotinaldehyde, methyl nicotinate, propyl nicotinate, 2-butoxyethyl nicotinate, benzyl nicotinate, 1-acetoxychavicol, polygodial or isodrimeninol.

Pungent-tasting plant extracts can be all plant extracts that are suitable for nutrition and cause a pungent or hot sensory impression. Preferred pungent-tasting plant extracts are, for example, pepper extract (Piper ssp., in particular Piper nigrum), water-pepper extract (Polygonum ssp., in particular Polygonum hydropiper), extracts of Allium ssp. (in particular onion and garlic extracts), extracts of radish (Raphanus ssp.), horse radish extracts (Cochlearia armoracia), extracts of black (Brassica nigra), wild or yellow mustard (Sinapis ssp., in particular Sinapis arvensis and Sinapis alba), pellitory root extracts (Anacyclus ssp., in particular Anacyclus pyrethrum L.), cone flower extracts (Echinaceae ssp.), extracts of Szechuan pepper (Zanthoxylum ssp., in particular Zanthoxylum piperitum), spilanthes extract (Spilanthes ssp., in particular Spilanthes acmella), chilli extract (Capsicum ssp., in particular Capsicum frutescens), grains of paradise extract (Aframomum ssp., in particular Aframomum melegueta [Rosc.] K. Schum.), ginger extract (Zingiber ssp., in particular Zingiber officinale) and galangal extract (Kaempferia galanga or Alpinia galanga).

The pungent-tasting plant extracts can be obtained from the corresponding fresh or dried plants or plant parts, but especially from white, green or black peppercorns, water pepper seeds, onions and garlic, radish roots, horse radish, mustard seeds, cone flower roots, pellitory root, plant parts of Zanthoxylum species, plant parts of spilanthes species, chilli peppers, grains of paradise or ginger or galangal roots, by extracting the dried plant parts, preferably comminuted in advance, with a solvent suitable for food and drink, but preferably ethanol, water, hexane or heptane, or ethanol/water mixtures, at 0° C. up to the boiling point of the respective solvent or mixture, then filtering and completely or partially concentrating the filtrate, preferably by distillation, freeze-drying or spray-drying. The resultant crude extract can then be worked up further, for example treated with steam at pressures from 0.01 mbar to atmospheric pressure and/or taken up in a solvent suitable for food and drink.

A solvent suitable for food and drink can be, for example: water, ethanol, methanol, propylene glycol, glycerol, acetone, dichloromethane, diethyl ether, hexane, heptane or supercritical carbon dioxide or mixtures of the above-mentioned solvents.

The invention also provides preparations consumed for nutrition or pleasure containing the mandelic acid alkylamides according to the invention in an effective quantity and optionally other conventional basic materials, auxiliary substances and additives for food and drink. They generally contain 0.000001 wt. % to 10 wt. %, preferably 0.0001 to 1 wt. %, but especially 0.0001 wt. % to 0.1 wt. %, based on the total weight of the preparation, of the mandelic acid alkylamides and mixtures thereof. Other conventional basic materials, auxiliary substances and additives for foods or drinks can be present in quantities of 0.000001 to 99.999999 wt. %, preferably 10 to 80 wt. %, based on the total weight of the preparation. In addition, the preparations can contain water in a quantity of up to 99.999999 wt. %, preferably 5 to 80 wt. %, based on the total weight of the preparation.

The preparations consumed for nutrition or pleasure in the context of the present invention are, for example, bakery products (e.g. bread, dry biscuits, cakes, other baked goods), confectionery (e.g. types of chocolate, fruit gums, hard and soft caramels, chewing gum), alcoholic or non-alcoholic beverages (e.g. coffee, tea, wine, wine-containing beverages, beer, beer-containing beverages, liqueurs, spirits, brandies, fruit-containing fizzy drinks, isotonic drinks, soft drinks, nectars, fruit and vegetable juices, fruit juice or vegetable juice preparations), instant drinks, meat products (e.g. ham, fresh sausage preparations or uncooked sausage preparations), eggs or egg products (dried egg, egg white, egg yolk), cereal products (e.g. breakfast cereals, muesli bars), milk products (e.g. milk drinks, dairy ice cream, yoghurt, kefir, fresh cheese, soft cheese, hard cheese, dried milk powder, whey, butter, buttermilk), fruit preparations (e.g. preserves, fruit ice, fruit sauces), vegetable preparations (e.g. ketchup, sauces, dried vegetables), snack products (for example baked or deep-fried potato crisps or potato dough products, maize- or peanut-based extrudates), fat- and oil-based products or emulsions thereof (e.g. mayonnaise, remoulade, dressings), prepared dishes and soups, spices, seasoning mixes and, in particular, seasonings for sprinkling, which are used in the snacks sector. The preparations within the meaning of the invention can also be used as semi-finished products for the production of further preparations consumed for nutrition or pleasure. The preparations within the meaning of the present invention can also be food supplements in the form of capsules, tablets (uncoated and coated tablets, for example coatings resistant to gastric juice), dragees, granules, pellets, solids mixtures, dispersions in liquid phases, as emulsions, as powders, as solutions, as pastes or as other preparations suitable for swallowing or chewing.

It has also proved to be particularly advantageous that the mandelic acid alkylamides according to the invention, especially the mandelic acid alkylamides according to the invention in combination with pungent-tasting plant extracts, can imitate the pungent taste of alcohol in alcoholic beverages or preparations of alcoholic beverages, and it is thus possible to lower the alcohol content in alcoholic beverages or in preparations of alcoholic beverages while maintaining the same sensory evaluation.

It has also proved to be particularly advantageous that the mandelic acid alkylamides according to the invention can imitate the pungent taste of capsaicin, dihydrocapsaicin and nonivamide and it is thus possible to lower the capsaicin content in preparations for use in nutrition, oral hygiene or consumed for pleasure, while maintaining the same sensory evaluation.

Another preferred embodiment of the invention is represented by preparations used for oral hygiene, especially dental care compositions such as toothpastes, tooth gels, toothpowders, mouthwashes, chewing gums and other oral care compositions containing the mandelic acid alkylamides in an effective quantity and optionally other conventional basic materials, auxiliary substances and additives for such preparations. They generally contain 0.000001 wt. % to 10 wt. %, preferably 0.0001 to 1 wt. %, but especially 0.0001 wt. % to 0.1 wt. %, based on the total weight of the preparation, of the mandelic acid alkylamides and mixtures thereof. Other conventional basic materials, auxiliary substances and additives for preparations used for oral hygiene can be contained in quantities of 0.000001 to 99.999999 wt. %, preferably 10 to 80 wt. %, based on the total weight of the preparation. In addition, the preparations can contain water in a quantity of up to 99.999999 wt. %, preferably 5 to 80 wt. %, based on the total weight of the preparation.

Dental care compositions containing the mandelic acid alkylamides according to the invention generally consist of an abrasive system (abrasives or polishes), such as e.g. silicas, calcium carbonates, calcium phosphates, aluminium oxides and/or hydroxylapatites, surface active substances, such as e.g. sodium lauryl sulphate, sodium lauryl sarcosinate and/or cocamidopropylbetaine, humectants, such as e.g. glycerol and/or sorbitol, thickeners, such as e.g. carboxymethylcellulose, polyethylene glycols, carrageenans and/or Laponites®, sweeteners, such as e.g. saccharin, stabilisers and active compounds, such as e.g. sodium fluoride, sodium monofluorophosphate, tin difluoride, quaternary ammonium fluorides, zinc citrate, zinc sulfate, tin pyrophosphate, tin dichloride, mixtures of various pyrophosphates, triclosan, cetylpyridinium chloride, aluminium lactate, potassium citrate, potassium nitrate, potassium chloride, strontium chloride, hydrogen peroxide, flavours and/or sodium bicarbonate.

Chewing gums containing the mandelic acid alkylamides according to the invention generally consist of a chewing gum base, that is to say a masticatory substance becoming plastic during chewing, sugars of various types, sugar substitutes, sweeteners, sugar alcohols, humectants, thickeners, emulsifiers, stabilisers and flavours.

The preparations according to the invention containing the mandelic acid alkylamides according to the invention can be prepared in that the mandelic acid alkylamides according to the invention are incorporated as the substance alone, as a solution or in the form of a mixture with a solid or liquid carrier into the preparations for use in nutrition, oral hygiene or consumed for pleasure.

To produce the preparations in another preferred embodiment, the mandelic acid alkylamides according to the invention and optionally other constituents of the preparation according to the invention can also be incorporated in advance into emulsions, into liposomes, e.g. starting from phosphatidyl choline, into microspheres, into nanospheres or else into capsules made of a matrix suitable for food and drinks, e.g. of starch, starch derivatives, other polysaccharides, natural fats, natural waxes or proteins, e.g. gelatin. Another embodiment consists in the fact that the mandelic acid alkylamides according to the invention are complexed in advance with suitable complexing agents, e.g. with cyclodextrins or cyclodextrin derivatives, preferably β-cyclodextrin, and used in this form.

Further constituents which can be used for the preparations according to the invention consumed for nutrition or pleasure are other conventional basic materials, auxiliary substances and additives for foods and drinks, e.g. water, mixtures of fresh or processed, vegetable or animal basic materials or raw materials (e.g. raw, fried, dried, fermented, smoked and/or cooked meat, egg, bone, cartilage, fish, crustaceans and shellfish, vegetables, fruits, herbs, nuts, vegetable or fruit juices or pastes or mixtures thereof), digestible or indigestible carbohydrates (e.g. sucrose, maltose, fructose, glucose, dextrins, amylose, amylopectin, inulin, xylans, cellulose), sugar alcohols (e.g. sorbitol, mannitol, xylitol), natural or hydrogenated fats (e.g. tallow, lard, palm kernel fat, coconut fat, hydrogenated vegetable fat), oils (e.g. sunflower oil, peanut oil, corn oil, thistle oil, olive oil, walnut oil, fish oil, soybean oil, sesame seed oil), fatty acids or their salts (e.g. potassium stearate, potassium palmitate), proteinogenic or non-proteinogenic amino acids and related compounds (e.g. taurine, creatine, creatinine), peptides, native or processed proteins (e.g. gelatin), enzymes (e.g. peptidases, glucosidases, lipases), nucleic acids, nucleotides (inositol phosphate), flavour-enhancing substances (e.g. monosodium glutamate, 2-phenoxypropionic acid), emulsifiers (e.g. lecithins, diacylglycerols), stabilisers (e.g. carageenan, alginate, locust bean gum, guar gum), preservatives (e.g. benzoic acid, sorbic acid), antioxidants (e.g. tocopherol, ascorbic acid), chelators (e.g. citric acid), organic or inorganic acidulants (e.g. malic acid, acetic acid, citric acid, tartaric acid, phosphoric acid), bitter substances (e.g. quinine, caffeine, limonin), sweeteners (e.g. saccharin, cyclamate, aspartame, neotame, neohesperidine dihydrochalcone), mineral salts (e.g. sodium chloride, potassium chloride, magnesium chloride, sodium phosphates), substances inhibiting enzymatic browning (e.g. sulfite, ascorbic acid), essential oils, plant extracts, natural or synthetic colours or colour pigments (e.g. carotenoids, flavonoids, anthocyans, chlorophyll and derivatives thereof), spices, as well as odour compounds and synthetic, natural or nature-identical flavour and taste compounds.

Preferably, the preparations according to the invention can also contain a flavour composition in order to round off and refine the taste and/or odour of the preparation. Suitable flavour compositions contain, for example, synthetic, natural or nature-identical flavour compounds and odour compounds, but especially also other pungent-tasting and/or heat-generating substances or plant extracts.

The invention also provides the use of the preparations according to the invention as semi-finished products for the flavouring of preparations made from them as finished products.

### EXAMPLES

Preparation of the Amides

The mandelic acid (2.53 mmol) and N-hydroxysuccinimide (2.53 mmol) are initially added to dry 1,4-dioxane (20 ml) under nitrogen and N,N′-dicyclohexylcarbodiimide (2.53 mmol) is added at ambient temperature. The mixture, which becomes cloudy, is stirred for 16 h at ambient temperature and filtered using a glass frit (P3). The filtrate is poured into a solution of the amine or ammonium hydrochloride (3.03 mmol) in water (10 ml) and NaHCO₃ (3.03 mmol) is added. The resulting mixture is stirred for 1.5 h at 50° C. and then brought to pH<2 using 5% hydrochloric acid, extracted 3 times with ethyl acetate, the combined organic phases are washed with saturated NaCl solution and optionally washed again with hydrochloric acid, dried over Na₂SO₄, filtered and concentrated by evaporation at 40° C./230-20 mbar. The residue is optionally chromatographed on silica gel 60 and/or recrystallised.

### Example 1

### 2-(4-Hydroxylphenyl)-2-hydroxy-N-heptylacetamide

Yield: 28% after recrystallisation; purity>98% (HPLC); HPLC-MS (APCI+) m/z=266.11 (100%, [M+H]⁺), 249.35 (84.6%, [M−HO+H]⁺), 530.61 (22.6%, [2M+H]⁺); ¹H-NMR (200 MHz; CD₃OD) δ=7.23 (2H, m, AA′) 6.74 (2H, m, BB′), approx. 4.9 ppm (s, below D₂O signal), 3.21 (2H, t, 7 Hz), 1.50 (2H, m), 1.40-1.20 (8H, m), 0.88 (3H, t, 7 Hz) ppm; ¹³C-NMR (50 Hz, CD₃OD) 175.68 (C), 158.36 (C), 132.55 (C), 129.15 (2×CH), 115.95 (2×CH), 75.12 (CH), 40.10 (CH₂), 39.98 (CH₂), 32.91 (CH₂), 30.49 (CH₂), 30.05 (CH₂), 27.82 (CH₂), 23.61 (CH₂), 14.43 (CH₃) ppm.

### Example 2

### 2-(4-Hydroxyphenyl)-2-hydroxy-N-octylacetamide

Yield: 52% after recrystallisation; purity>95% (HPLC); HPLC-MS (APCI+) m/z=280.07 (100%, [M+H]⁺), 262.61 (84. %, [M−H₂O+H]⁺), 558.68 (15.3%, [2M+H]⁺); ¹H-NMR (200 MHz; CD₃OD) δ=7.23 (2H, m, AA′) 6.74 (2H, m, BB′), approx. 4.89 ppm (s, below D₂O signal), 3.21 (2H, t, 7 Hz), 1.50 (2H, m), 1.40-1.20 (10H, m), 0.89 (3H, t, 7 Hz) ppm; ¹³C-NMR (50 Hz, CD₃OD) 175.60 (C), 158.36 (C), 132.55 (C), 129.15 (2×CH), 115.95 (2×CH), 75.11 (CH), 40.10 (CH₂), 39.98 (CH₂), 32.91 (CH₂), 30.48 (CH₂), 30.34 (2×CH₂), 27.87 (CH₂), 23.68 (CH₂), 14.44 (CH₃) ppm.

### Example 3

### 2-(4-Hydroxyphenyl)-2-hydroxy-N-nonylacetamide

Yield: 76% after recrystallisation; purity>90% (HPLC); HPLC-MS (APCI+) m/z=294.10 (100%, [M+H]⁺), 276.51 (61.8%, [M−H₂O+H]⁺), 586.75 (6.45%, [2M+H]⁺); ¹H-NMR (200 MHz; CD₃OD) δ=7.22 (2H, m, AA′) 6.71 (2H, m, BB′), 4.89 ppm (s, below D₂O signal), 3.21 (2H, t, 7 Hz), 1.50 (2H, m), 1.40-1.20 (12H, m), 0.88 (3H, t, 7 Hz) ppm; ¹³C-NMR (50 Hz, CD₃OD) 175.59 (C), 158.36 (C), 132.54 (C), 129.15 (2×CH), 115.95 (2×CH), 75.09 (CH), 39.98 (CH₂), 32.99 (CH₂), 30.63 (CH₂), 30.47 (CH₂), 30.38 (CH₂), 30.34 (CH₂), 27.87 (CH₂), 23.69 (CH₂), 14.45 (CH₃) ppm.

### Example 4

### 2-(3,4-Dihydroxyphenyl)-2-hydroxy-N-octylacetamide

Yield: 16% after chromatography; HRMS (direct inlet): measured 295.17650, calculated 295.17834 for C₁₆H₂₅NO₄; HPLC-MS (ESI+) m/z=277.95 (100%, [M-H₂O+H]⁺), 295.80 (92.76%, [M+H]⁺), 590.63 (23.3%, [2M+H]⁺); ¹H-NMR (200 MHz; CD₃OD) δ=6.84 (1H, m), 6.73-6.71 (2H, m), 4.84 ppm (1H, s), 3.21 (2H, t, 7 Hz), 1.50 (2H, m), 1.40-1.20 (10H, m), 0.89 (3H, t, 7 Hz) ppm; ¹³C-NMR (50 Hz, CD₃OD) 175.57 (C), 146.25 (C), 146.09 (C), 133.14 (C), 119.61 (CH), 115.93 (CH), 115.08 (CH), 75.22 (CH), 40.03 (CH₂), 32.92 (CH₂), 30.49 (CH₂), 30.35 (2×CH₂), 27.90 (CH₂), 23.68 (CH₂), 14.45 (CH₃) ppm.

### Example 5

### 2-(3-Hydroxy-4-methoxyphenyl)-2-hydroxy-N-heptylacetamide

Yield: 47% after chromatography; purity>95% (HPLC); HPLC-MS (APCI+) m/z=278.51 (100%, [M+H−H₂O]⁺), 295.97 (84.6%, [M+H]⁺), 590.81 (20.4%, [2M+H]⁺); HRMS (direct inlet): measured 295.17699, calculated 295.17834 for C₁₆H₂₅NO₄; ¹H-NMR (400 MHz; CD₃OD; with water suppression) δ=6.89 (1H, s), 6.86 (2H, m), approx. 4.9 ppm (suppressed), 3.83 (3H, s), 3.22 (2H, td, 7 Hz, 1 Hz), 1.51 (2H, tt, 7 Hz, 7 Hz), 1.35-1.22 (8H, m), 0.90 (3H, t, 7 Hz) ppm; ¹³C-NMR (100 Hz, CD₃OD) 175.67 (C), 149.03 (C), 147.59 (C), 134.75 (C), 119.56 (CH), 114.97 (CH), 112.41 (CH₂), 75.25 (CH), 56.43 (CH₃), 40.08 (CH₂), 32.99 (CH₂), 30.57 (CH₂), 30.14 (CH₂), 27.90 (CH₂), 23.67 (CH₂), 14.46 (CH₃) ppm.

### Example 6

### 2-(3-Hydroxy-4-methoxyphenyl)-2-hydroxy-N-octylacetamide

Yield: 36% after chromatography; purity>95% (HPLC); HPLC-MS (APCI+) m/z=292.66 (100%, [M+H H₂O]⁺), 310.01 (69.6%, [M+H]⁺), 618.83 (46%, [2M]⁺); HRMS (direct inlet): measured 309.19519, calculated 309.19400 for C₁₇H₂₇NO₄; ¹H-NMR (400 MHz; CD₃OD; with water suppression) δ=6.88 (1H, m), 6.85 (2H, m), approx. 4.9 ppm (suppressed), 3.83 (3H, s), 3.21 (2H, td, 7 Hz, 1 Hz), 1.50 (2H, m), 1.40-1.22 (10H, m), 0.88 (3H, t, 7 Hz) ppm.

### Example 7

### 2-(3-Hydroxy-4-methoxyphenyl)-2-hydroxy-N-nonylacetamide

Yield: 50%, colourless crystals; purity>95% (HPLC); HPLC-MS (APCI+) m/z=324.16 (100%, [M+H]⁺), 306.60 (80%, [M+H−H₂O]⁺), 646.88 (47%, [2M+H]⁺); HRMS (direct inlet): measured 323.20822, calculated 323.20966 for C₁₈H₂₉NO₄; ¹H-NMR (200 MHz; CD₃OD) δ=6.88 (1H, m), 6.86 (2H, m), 4.87 ppm (1H, s), 3.82 (3H, s), 3.21 (2H, t, 7 Hz), 1.51 (2H, m), 1.40-1.25 (12H, m), 0.88 (3H, t, 7 Hz) ppm.

### Example 8

### 2-(4-Hydroxy-3-methoxyphenyl)-2-hydroxy-N-heptylacetamide

Yield: quantitative; purity>95% (HPLC); HPLC-MS (APCI+) m/z=278.61 (100%, [M+H−H₂O]⁺), 296.17 (27%, [M+H]⁺), 590.78 (12%, [2M+H]⁺); HRMS (direct inlet): measured 295.17679, calculated 295.17834 for C₁₆H₂₅NO₄; ¹H-NMR (200 MHz; CD₃OD; with water suppression) δ=7.00 (1H, d, 2 Hz), 6.86 (1H, dd, 8 Hz, 2 Hz), 6.74 (1H, d, 8 Hz), approx. 4.9 ppm (suppressed), 3.83 (3H, s), 3.21 (2H, td, 7 Hz, 1 Hz), 1.50 (2H, m), 1.40-1.20 (12H, m), 0.89 (3H, t, 7 Hz) ppm; ¹³C-NMR (50 Hz, CD₃OD) 175.60 (C), 148.74 (C), 147.49 (C), 133.17 (C), 120.76 (CH), 115.82 (CH), 111.34 (CH), 75.36 (CH), 56.27 (CH₃), 40.01 (CH₂), 32.96 (CH₂), 30.55 (CH₂), 30.11 (CH₂), 27.87 (CH₂), 23.65 (CH₂), 14.44 (CH₃) ppm.

### Example 9

### 2-(4-Hydroxy-3-methoxyphenyl)-2-hydroxy-N-octylacetamide

Yield: 76% after chromatography; purity>95% (HPLC); HPLC-MS (APCI+) m/z=292.40 (100%, [M+H−H₂O]⁺), 309.90 (31%, [M+H]⁺), 618.71 (5%, [2M+H]⁺); HRMS (direct inlet): measured 309.19209, calculated 309.19400 for C₁₇H₂₇NO₄; ¹H-NMR (200 MHz; CD₃OD) δ=7.01 (1H, d, 2 Hz), 6.88 (1H, dd, 8 Hz, 2 Hz), 6.76 (1H, d, 8 Hz), approx. 4.9 ppm (below the HDO signal), 3.85 (3H, s), 3.21 (2H, td, 7 Hz, 1 Hz), 1.51 (2H, m), 1.38-1.22 (10H, m), 0.89 (3H, t, 7 Hz) ppm; ¹³C-NMR (50 Hz, CD₃OD) 175.60 (C), 148.76 (C), 147.50 (C), 133.19 (C), 120.78 (CH), 115.83 (CH), 111.37 (CH), 75.36 (CH), 56.29 (CH₃), 40.02 (CH₂), 32.96 (CH₂), 30.56 (CH₂), 30.41 (2□CH₂), 27.92 (CH₂), 23.73 (CH₂), 14.45 (CH₃) ppm.

### Example 10

### 2-(4-Hydroxy-3-methoxyphenyl)-2-hydroxy-N-nonylacetamide

Yield: 1.5 g after chromatography; purity>95% (HPLC); HPLC-MS (APCI+) m/z=306.43 (100%, [M+H−H₂O]⁺), 323.21 (22.5%), 324.08 (21.8%, [M+H]⁺); HRMS (direct inlet): measured 323.20855, calculated 323.20966 for C₁₈H₂₉NO₄; ¹H-NMR (200 MHz; CD₃OD; with water suppression) δ=7.01 (1H, d, 2 Hz), 6.88 (1H, dd, 8 Hz, 2 Hz), 6.74 (1H, d, 8 Hz), approx. 4.9 ppm (suppressed), 3.85 (3H, s), 3.22 (2H, td, 7 Hz, 1 Hz), 1.60-1.40 (2H, m), 1.40-1.20 (12H, m), 0.89 (3H, t, 7 Hz) ppm; ¹³C-NMR (50 Hz, CD₃OD) 175.57 (C), 148.72 (C), 147.47 (C), 133.14 (C), 120.75 (CH), 115.81 (CH), 111.33 (CH), 75.33 (CH), 56.26 (CH₃), 39.99 (CH₂), 33.04 (CH₂), 30.68 (CH₂), 30.53 (CH₂), 30.43 (CH₂), 30.38 (CH₂), 27.91 (CH₂), 23.72 (CH₂), 14.47 (CH₃) ppm.

### Example 11

### Tasting the Mandelic Acid Alkylamides

The substance to be tasted is dissolved in ethanol and the ethanolic solution is then diluted with an 11% sugar solution (final concentration: c). For tasting, in each case approximately 5 ml of the sugar solution are swallowed. If the threshold value of the substance is known, a value just above the threshold value is chosen for the tasting. A group of 6-8 testers tasted the solutions.

Where possible, the impression of pungency was estimated on a scale of 1 (very weak) to 9 (very strong).

a) Profile of 2-(3,4-dihydroxyphenyl)-2-hydroxy-N-octylacetamide (no. 4):

10 ppm: pungency develops slowly; stinging, rough, slightly spicy; estimation of pungency: 5.
   b) Profile of 2-(3-hydroxy-4-methoxyphenyl)-2-hydroxy-N-octylacetamide (no. 6):
1 ppm: slightly pungent, particularly on the tongue, slight tingling, rapidly disappears again; estimation of pungency: 3.
   c) Profile of 2-(4-hydroxy-3-methoxyphenyl)-2-hydroxy-N-heptylacetamide (no. 8):
10 ppm: pungency develops; burning, slightly over-ripe; estimation of pungency: 7.
   d) Profile of 2-(4-hydroxy-3-methoxyphenyl)-2-hydroxy-N-octylacetamide (no. 9):
1 ppm: no pungency initially, develops suddenly, very intense and long-lasting, ginger pungency; estimation of pungency: 8.
   e) Profile of 2-(4-hydroxy-3-methoxyphenyl)-2-hydroxy-N-nonylacetamide (no. 10):
10 ppm: immediate onset of pungency; similar to capsaicin; estimation of pungency: 8.

### Comparative Examples

f) Profile of Dihydrocapsaicin:

100 ppb: slightly delayed onset of effect in pharyngeal cavity, burning, aggressive, no development of heat.
   g) Profile of N-(3-methoxy-4-hydroxybenzyl)nonanoamide
200 ppb: slightly delayed onset of effect in pharyngeal cavity, little pungency on the tongue, stinging, no development of heat

### Example 12

### Use as a Flavour Compound in a Toothpaste

| | | Quantity |
|---|---|---|
| | | used in |
| Component | Constituent | wt. % |
| A | Demineralised water | 22.00 |
| | Sorbitol (70%) | 45.00 |
| | Solbrol ® M, sodium salt (Bayer AG, alkyl | 0.15 |
| | p-hydroxybenzoate) | |
| | Trisodium phosphate | 0.10 |
| | Saccharin, 450 fold | 0.20 |
| | Sodium monofluorophosphate | 1.12 |
| | Polyethylene glycol 1500 | 5.00 |
| B | Sident 9 (abrasive silicon dioxide) | 10.00 |
| | Sident 22 S (thickening silicon dioxide) | 8.00 |
| | Sodium carboxymethylcellulose | 0.90 |
| | Titanium dioxide | 0.50 |
| C | Demineralised water | 4.53 |
| | Sodium lauryl sulfate | 1.50 |
| D | Flavour, containing 0.1% 2-(4-hydroxy-3- | 1 |
| | methoxyphenyl)-2-hydroxy-N- | |
| | octylacetamide | |

The constituents of components A and B are premixed for each component separately and stirred well together under vacuum for 30 min at 25-30° C. Component C is premixed and added to A and B; D is added and the mixture is stirred well under vacuum at 25-30° C. for 30 min. After release of vacuum, the toothpaste is finished and can be packaged.

### Example 13

### Use as a Flavour Compound in a Sugar-Free Chewing Gum

| | | Quantity |
|---|---|---|
| | | used in |
| Component | Constituent | wt. % |
| A | Chewing gum base, company “Jagum T” | 30.00 |
| B | Sorbitol, powdered | 39.00 |
| | Isomalt ® (Palatinit GmbH) | 9.50 |
| | Xylitol | 2.00 |
| | Mannitol | 3.00 |
| | Aspartame ® | 0.10 |
| | Acesulfame ® K | 0.10 |
| | Emulgum ® (Colloides Naturels, Inc.) | 0.30 |
| C | Sorbitol, 70% | 14.00 |
| | Glycerol | 1.00 |
| D | Flavour, containing 0.1% 2-(4-hydroxy-3- | 1 |
| | methoxyphenyl)-2-hydroxy-N-heptylacetamide | |

Components A to D are mixed and kneaded intensively. The raw mass can be processed in the form of thin strips, for example, to give ready-to-eat chewing gums.

### Example 14

### Use as a Flavour Compound in a Mouthwash

| | | Content |
|---|---|---|
| Component | Constituent | (%) |
| A | Ethanol | 10.00 |
| | Cremophor ® CO 40 (BASF, Detergenz) | 1.00 |
| | Benzoic acid | 0.12 |
| | Flavour, containing 0.4% 2-(4-hydroxy-3- | 0.25 |
| | methoxyphenyl)-2-hydroxy-N-nonylacetamide | |
| B | Demineralised water | 83.46 |
| | Sorbitol, 70% | 5.00 |
| | Sodium saccharin 450 | 0.07 |
| | L-Blue 5000 e.c., 1% in water (colour) | 0.10 |

The constituents of components A and B are each mixed separately. Component B is slowly stirred into component A until the mixture is homogeneous.

### Example 15

### Use in Combination with a Pungent Plant Extract as an Alcohol Intensifier

Comparative sample: Liqueur base 10 vol. %

| | | |
|---|---|---|
| | 7.39 kg | alcohol, reagent grade |
| | 20 kg | invert sugar syrup, 66.5% solids |
| | 72.61 kg | water |
| Total | 100 kg | |

Liqueur base 5.5 vol. %

| | | |
|---|---|---|
| | 4.06 kg | alcohol, reagent grade |
| | 20 kg | invert sugar syrup, 66.5% solids |
| | 75.94 kg | water |
| Total | 100 kg | |

Version A: Liqueur base 5.5 vol. %+0.3% of a 10% solution of a grains of paradise extract in ethanol.

Version B: Liqueur base 5.5 vol. %+0.075% of a 10% solution of a grains of paradise extract in ethanol+0.02% of a solution of 1% 2-(4-hydroxy-3-methoxyphenyl)-2-hydroxy-N-nonylacetamide in ethanol (corresponding to 2 ppm).

The alcoholic pungency of the comparative sample is imitated better in version B than in version A from a sensory point of view. The sensory evaluations of version A and the comparative sample are very similar.

1. A flavouring composition comprising a mandelic acid alkylamide of general formula (I) 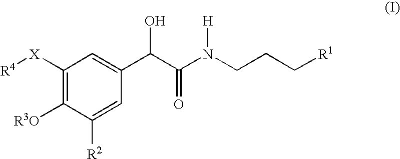
wherein
X represents a single bond or an oxygen atom, and
R¹ represents a linear or branched alkyl residue with 1 to 20 carbon atoms or a linear or branched alkenyl residue with 2 to 20 carbon atoms, and
R² represents a hydrogen atom, a hydroxy group or an O—R⁵ group, and
R³, R⁴ and R⁵, independently of one another, represent hydrogen or a lower alkyl residue or a lower alkenyl residue, or
R³ and R⁴ together represent a —CR⁶R⁷— group,
and R⁶ and R⁷, independently of one another, represent hydrogen or lower alkyl residues or lower alkenyl residues,
and stereoisomers or mixtures thereof.

2. A flavouring composition comprising
2-(4-hydroxyphenyl)-2-hydroxy-N-heptylacetamide,
2-(4-hydroxyphenyl)-2-hydroxy-N-octylacetamide,
2-(4-hydroxyphenyl)-2-hydroxy-N-nonylacetamide,
2-(4-methoxyphenyl)-2-hydroxy-N-heptylacetamide,
2-(4-methoxyphenyl)-2-hydroxy-N-octylacetamide,
2-(4-methoxyphenyl)-2-hydroxy-N-nonylacetamide,
2-(3,4-dihydroxyphenyl)-2-hydroxy-N-octylacetamide,
2-(3-hydroxy-4-methoxyphenyl)-2-hydroxy-N-heptylacetamide,
2-(3-hydroxy-4-methoxyphenyl)-2-hydroxy-N-octylacetamide,
2-(3-hydroxy-4-methoxyphenyl)-2-hydroxy-N-nonylacetamide,
2-(4-hydroxy-3-methoxyphenyl)-2-hydroxy-N-heptylacetamide,
2-(4-hydroxy-3-methoxyphenyl)-2-hydroxy-N-octylacetamide,
2-(4-hydroxy-3-methoxyphenyl)-2-hydroxy-N-nonylacetamide, and
2-(4-hydroxy-3-methoxyphenyl)-2-hydroxy-N-(7-methyl-1-octyl)acetamide
and stereoisomers or mixtures thereof.

3. A flavouring composition according to claim 1 exhibiting a pungent flavour or a flavour with a heat-generating effect.

4. A preparation consumed for nutrition or pleasure comprising a flavouring composition according to claim 1.

5. An oral hygiene composition comprising a flavouring composition according to claim 1.

6. Preparations for use in nutrition, oral hygiene or consumed for pleasure containing mandelic acid alkylamides of general formula (I) 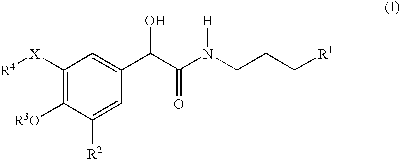
wherein
X represents a single bond or an oxygen atom, and
R¹ represents a linear or branched alkyl residue with 1 to 20 carbon atoms or a linear or branched alkenyl residue with 2 to 20 carbon atoms, and
R² represents a hydrogen atom, a hydroxy group or an O—R⁵ group, and
R³, R⁴ and R⁵, independently of one another, represent hydrogen or a lower alkyl residue or a lower alkenyl residue, or
R³ and R⁴ together represent a —CR⁶R⁷— group, and
R⁶ and R⁷, independently of one another, represent hydrogen or lower alkyl residues or lower alkenyl residues, and stereoisomers or mixtures thereof.

7. Preparations according to claim 6, containing at least one other pungent-tasting or heat-generating substance.

8. Preparations according to claim 6, containing at least one pungent-tasting plant extract.

9. Preparations according to claim 6, containing at least one other pungent-tasting or heat-generating substance and at least one pungent-tasting plant extract.

10. Preparations according to claim 6 in the form of a semi-finished product.

11. Preparations according to claim 6 in the form of odour, flavour and taste compositions and seasoning mixes.

12. Mandelic acid alkylamide flavouring compounds of general formula (I) 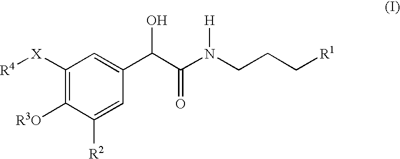
wherein
R¹ represents a linear or branched alkyl residue with 1 to 20 carbon atoms or a linear or branched alkenyl residue with 2 to 20 carbon atoms and
R² represents a hydrogen atom, and, either
X represents a single bond,
R³ a lower alkyl residue or a lower alkenyl residue and
R⁴ hydrogen, or
X represents an oxygen atom,
R³ hydrogen, and
R⁴ a lower alkyl residue or a lower alkenyl residue, or
X represents an oxygen atom,
R³ a lower alkyl residue or a lower alkenyl residue, and
R⁴ hydrogen,
and stereoisomers or mixtures thereof with the exception that X represents an oxygen atom, R¹ 1-pentyl, R² and R³ hydrogen and R⁴ methyl.

13. 2-(4-Hydroxyphenyl)-2-hydroxy-N-heptylacetamide,
2-(4-hydroxyphenyl)-2-hydroxy-N-octylacetamide,
2-(4-hydroxyphenyl)-2-hydroxy-N-nonylacetamide,
2-(4-methoxyphenyl)-2-hydroxy-N-heptylacetamide,
2-(4-methoxyphenyl)-2-hydroxy-N-octylacetamide,
2-(4-methoxyphenyl)-2-hydroxy-N-nonylacetamide,
2-(3-hydroxy-4-methoxyphenyl)-2-hydroxy-N-heptylacetamide,
2-(3-hydroxy-4-methoxyphenyl)-2-hydroxy-N-nonylacetamide,
2-(4-hydroxy-3-methoxyphenyl)-2-hydroxy-N-heptylacetamide,
2-(4-hydroxy-3-methoxyphenyl)-2-hydroxy-N-nonylacetamide, and
2-(4-hydroxy-3-methoxyphenyl)-2-hydroxy-N-(7-methyl-1-octyl)acetamide.

14. A process for producing the flavouring compounds according to claim 12, characterised in that a mandelic acid of general formula II 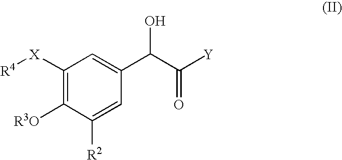
wherein
X, R², R³ and R⁴ have the meaning given in claim 12, and
Y represents an activated nucleofuge, or derivatives, the OH groups of which are protected with protective groups,
is reacted with an alkylamine of general formula (IIIa) 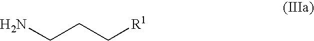
or an alkylammonium salt of general formula (IIIb) 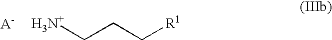
wherein R¹ has the meaning given above and A⁻ denotes an inorganic or organic anion.

15. (canceled)

16. Cosmetic or dermatological preparations containing mandelic acid alkylamides of general formula (I) 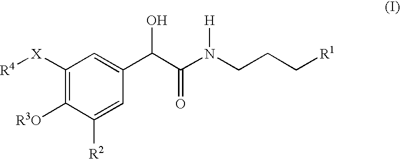
wherein
X represents a single bond or an oxygen atom and
R¹ represents a linear or branched alkyl residue with 1 to 20 carbon atoms or a linear or branched alkenyl residue with 2 to 20 carbon atoms and
R² represents a hydrogen atom, a hydroxy group or an O—R⁵ group and
R³, R⁴ and R⁵, independently of one another, represent hydrogen or a lower alkyl residue or a lower alkenyl residue, or R³ and R⁴ together represent a —CR⁶R⁷— group and
R⁶ and R⁷, independently of one another, represent hydrogen or lower alkyl residues or lower alkenyl residues,
and stereoisomers or mixtures thereof.
